(19) United States
(12) Patent Application Publication
(10) Pub. No.: US 2013/0165679 A1
(43) Pub. Date: Jun. 27, 2013

(54) ALPHA-GLUCOSIDASE INHIBITOR

(71) Applicants: Lean Teik NG, Taipei City (TW); Chun-Han Su, Taipei City (TW); Min-Nan Lai, Nantou County (TW)

(72) Inventors: Lean Teik NG, Taipei City (TW); Chun-Han Su, Taipei City (TW); Min-Nan Lai, Nantou County (TW)

(73) Assignee: KANG JIAN BIOTECH CORP., LTD., Caotun Town (TW)

(21) Appl. No.: 13/425,819

(22) Filed: Mar. 21, 2012

(30) Foreign Application Priority Data
Dec. 22, 2011 (TW) 100147931

(51) Int. Cl. C07C 57/03

(52) U.S. Cl. 554/224; 554/223

(57) ABSTRACT

A kind of α-glucosidase inhibitor, composing of mainly an unsaturated fatty acid composition, that acts to inhibit the decomposition of starch and disaccharides in the small intestine as well as slowing down the absorption of glucose, and thus achieving the objective of its application in preventing or treating diabetes and obesity.

### FIELD OF THE INVENTION

The present invention relates to a α-glucosidase inhibitor that acts to decrease the level of glucose in blood, and thus achieving the objective of its application in preventing or treating diabetes and obesity. Besides, the α-glucosidase inhibitor of the invention can also be used as a nutrient for human body.

### BACKGROUND OF THE INVENTION

Diabetes is a common endocrine metabolism disorder. It is also a global pandemic disease with increasing number of patients. Epidemiological investigations revealed that diabetes has become another serious chronic disease to human health, after cardiovascular diseases and cancer. According to statistics, diabetes causes numerous damages to human body. If blood sugar level in diabetic patients is not effectively controlled, in a long-term it can lead to lesions in many organs and tissues, in which heart brain, kidney, blood vessels, nerves and skin are the main sites of damage.

The types of diabetes include:

### 1. Type 1 Diabetes Mellitus (T1DM)

It is also called insulin-dependent diabetes mellitus (IDDM), and is due to destruction of pancreatic B cells, of which the cause remains unknown. Congenital genetic defects are believed to be the major factor, which result in the cell-mediated autoimmune response produced by immune system that attacks islet β-cells. In other words, the immune system of IDDM patient produces autoimmune antibody against islet β-cells, which are seriously damaged and thus can not function normally to secrete sufficient insulin for carbohydrate metabolism. This type of diabetes usually occurs in childhood. The level of insulin in diabetic patient is much lower than that in normal individual, this makes the patient must receive continuous insulin injections to control blood sugar throughout his or her whole life.

### 2. Type 2 Diabetes Mellitus (T2DM)

It is also called noninsulin-dependent diabetes mellitus (NIDDM), and is usually associated with acquired factors such as family heredity or obesity. NIDDM mostly occurs in the elderly people of age over 40. It comprises an array of dysfunctions resulting from the combination of resistance to insulin action (IR) and/or inadequate insulin secretion. There are two possible reasons that may lead to IR:
(a) The interrupted signaling occurred after the binding of insulin to receptor: in this case, insulin can bind to the receptor normally, but cells can not receive proper messages to implement carbohydrate metabolism.
(b) The defective insulin receptors on cells: insulin can not bind to the receptor, or the number of receptors is insufficient, hence insulin is unable to bind to the receptor to function properly.

The occurrence and progression of hyperglycemia symptom in Type 2 diabetes mellitus are very slow, which usually need several years to display obvious symptoms. Therefore, Type 2 diabetes mellitus is relatively hard to be detected. However, the symptoms of Type 2 diabetes is mellitus can be controlled by regular diet and exercise, without needs of insulin therapy.

### 3. Gestational Diabetes Mellitus (G.D.M)

It occurs during pregnancy. The level of blood sugar in patients with this type of diabetes mellitus will return to normal after parturition. However, the risk of re-occurring of abnormal blood glucose increases significantly.

### 4. Other Types of Diabetes Mellitus

Hyperglycemia is caused by various factors, including genetic defects of β cells, genetic defects in insulin function, pancreatic exocrine diseases, endocrine diseases, drug or chemical therapy, infection, rare immune-mediated disorders, and other genomic syndrome.

Among the different types of diabetes mellitus mentioned above. T1DM (accounted for about 5-10% of the diabetic population) and T2DM (about 90-95% of the diabetic population) are the two predominant diabetes mellitus, while the occurrence of other types of diabetes are relatively rare.

Currently, the methods used for treating the various types of diabetes can he classified into oral hypoglycemic drugs and insulin injection therapy, in which the oral hypoglycemic drugs can be further divided into four categories according to their mechanism of action:
1. Insulin secretagogues, such as sulfonylureas (SU), act on sulfonylurea receptor to increase insulin secretion; and glinides, act as stimulator of sulfonylurea receptor on β cells to increase the secretion of insulin.
2. Biguanides, such as Dimethyl biguanide, reduce the gluconeogenesis in liver without increasing insulin secretion.
3. Glitazones (Thiazolidinediones) act on activating peroxisome proliferator-activated receptor-γ (PPAR-γ) to increase the sensitivity to insulin, and thus reduce the fasting blood glucose level and the blood insulin concentration.
4. α-Glucosidase inhibitor, such as acarbose. voglibose, miglitol and the like, act on inhibiting the decomposition of starch and disaccharides in small intestine to delay the absorption of glucose, and therefore reduce the blood glucose level.

The above mentioned α-glucosidase inhibitors are commonly used as oral hypoglycemic drugs for treating a large proportion T2DM patients. The detailed mechanism of action of these drugs is described below :

After carbohydrate intake, polysaccharides are first decomposed into oligosaceharides or disaccharides by amylase in saliva or digestive enzymes (amylase) in pancreas. In small intestine, oligosaccharides are hydrolyzed to monosaccharides, such as glucose and fructose, by α-glucosidase secreted by intestine epithelial cells. Only the monosaccharides can enter into the blood circulation system and be utilized by human body. α-Glucosidase acts at the last step in the process of carbohydrate digestion. Accordingly, an effective inhibition of said enzyme may prevent the absorption of carbohydrate, and further inhibit the occurrence of postprandial hyperglycemia. Thus, inhibition of α-glucosidase activity is a good strategy for the prevention or treatment of diabetes mellitus, hyperlipoproteinemia and obesity.

Acarbose is a commercially available hypoglycemic agent. which is produced by the fermentation of Actinoplanes utahensis, and has a chemical structure similar to sugars. Acarbose reversibly competes with α-glucosidase and thus delays the conversion of disaccharides to monosaccharides, thereby to achieve the effect of lowering blood glucose. Currently acarbose is widely used as an effective oral hypoglycemic agent for treating T2DM.

Besides acarbose. α-glucosidase inhibitors that have been widely used include voglibose. miglitol and the like. However, in taking these hypoglycemic drugs, patients still encounter serious side effects, such as abdominal distention, abdominal pain, diarrhea, gastrointestinal spasmodic pain, constipation, bowel gurgling, nausea, vomiting, anorexia, fatigue, headache, dizziness, skin itching and others.

In view of the fact that the hypoglycemic treatment with α-glucosidase inhibitors remain having side effects as mentioned above, suggesting that the presently available drugs are harmful to the human body in long-term usage. With the advancement of biotechnology and pharmaceutical technology, and through many years of research and experiments, the inventors found that unsaturated fatty acids can act as a good α-glucosidase inhibitor. The present invention also discovered that the unsaturated fatty acids in the oily component of mushrooms, plants or animals are effective in controlling blood sugar, and can also be used as functional nutrients for preventing diabetes mellitus.

### SUMMARY OF THE INVENTION

In one aspect, the present invention provides a use of unsaturated fatty acids to act as a α-glucosidase inhibitor, and the application thereof in preventing or treating diabetes. The unsaturated fatty acids of the invention act in a different way from the current commercially available hypoglycemic drug acarbose, for that acarbose is a fermentation product of Actinoplanes utahensis, which has a structure similar to sugars and can delays the conversion of disaccharides to monosaccharides by reversible competition of α-glucosidase, and thus to achieve the effect of lowering blood glucose.

In other words, the traditional competitive inhibition occurs in the substrate binding site or the catalytic site; the substrate analog inhibitor can bind to the enzyme and form an enzyme-inhibitor complex without generating an enzyme-substrate complex because of its similar structure to the substrate. When the substrate and inhibitor are present simultaneously, they will produce competitive binding to the active site on enzyme surface: so acarbose is currently used as an oral hypoglycemic agent to treat Type 2 diabetes mellitus (T2DM.).

Although the unsaturated fatty acids of the invention are not structurally similar to carbohydrates, their action on the enzyme also belongs to the competitive inhibition. That is, the inhibitor may bind to a specific binding site of the enzyme. This inhibitor did not affect the Vmax but increased the Michaelis constant (Km). The major purpose of the invention is the use of one or more unsaturated fatty acids alone or in combination through oral or intravenous administration to prevent or to treat diabetes.

The unsaturated fatty acids of the invention can also be used as essential nutrients for human body, in addition to act as the α-glucosidase inhibitor. In general, unsaturated fatty acids are essential fatty acids to human body, so they would not produce side effects as traditional oral hypoglycemic agents do. According to the number of double bonds, unsaturated fatty acids can be divided into monounsaturated fatty acids and polyunsaturated fatty acids.

So far the naturally occurring essential fatty acids include: linoleic acid (Ω6), α-linolenic acid (Ω3), and arachidonic acid. In fact, arachidonic acid, eicosapentaenoic acid (EPA), docosahexaenoic acid (DHA) are also the essential fatty acids. however, they can be synthesized from linoleic acid and/or α-linolenic acid by human body.

The common important physiological functions of the above essential fatty acids on human body include: (1) essential fatty acids are the constituent of tissue cells; (2) cholesterol must be combined with essential fatty acids for its function and normal metabolism in the body: (3) essential fatty acids can protect the skin against radiation damages; (4) essential fatty acids benefit to pregnancy and lactation; and (5) essential fatty acids maintain normal growth and development of infants. Therefore, in another aspect, this invention features the use of the α-glucosidase inhibitor (unsaturated fatty acids) as a nutrient for human body.

### DETAILED DESCRIPTION OF THE INVENTION

The present invention relates to a use of unsaturated fatty acid as the α-glucosidase inhibitor. The following examples describe the evaluation of enzyme inhibition of unsaturated fatty acids to act as a α-glucosidase inhibitor, including the comparative experiments with traditional α-glucosidase inhibitors.

The specific examples below are to be construed as merely illustrative, and not limitative of the remainder of the disclosure in any way whatsoever. Without further elaboration, it is believed that one skilled in the art can. based on the description herein, utilize the present invention to its fullest extent. All publications cited herein arc hereby incorporated by reference in their entirety. Further, any mechanism proposed below does not in any way restrict the scope of the claimed invention.

### Example 1

Examples of unsaturated fatty acid used to analyze its enzyme inhibitive activity as a α-glucosidase inhibitor include oleic acid (Ω9), linoleic acid (Ω6), and α-linolenic acid (Ω3). Chemicals used in the analysis include: α-glucosidase (yeast. EC 3.2.1.20), α-linolenic acid and acarbose, purchased from Sigma-Aldrich Co. (Saint Louis, Mo. USA); oleic acid, purchased from Shown Co. (Tokyo, Japan); linoleic acid, purchased from MP Biomedicals Inc. (Aurora. Ohio, USA); and p-nitrophenyl-α-D-glucopyranoside purchased from Acros Organics Co, (Morris Plains, N.J., USA).

In the analytical method, p-nitrophenyl-α-D-glucopyranoside was used as a substrate. The absorbance was measured by spectrophotometry to determine the α-glucosidase inhibition activity of samples (Apostolidis E. Lee C M. In vitro potential of Ascophyllum nodosam phenolic antioxidant-mediated alpha-glucosidase and alpha-amylase inhibition. Journal of Food Science 2010, 75:H97-102; Kang W Y, Song Y L, Zhang L. α-Glucosidase inhibitory and antioxidant properties and antidiabetic activity of Hypericum ascyron L. Medicinal Chemistry Research 2011.20:809-816).

To a 1.5 ml eppendorf tube containing 0.05 ml of sample solution (in 5% methanol), 0.1 ml of α-glucosidase solution (2 U/ml; in 100 mM phosphate buffer, pH 6.9) was added. The mixture was incubated in 37° C. water bath for 10 min, and then 0.05 ml of 5 mM p-nitrophenyl-α-D-glucopyranoside (in 100 mM phosphate buffer, pH 6.9) was added and mixed homogeneously. The mixture was incubated in 37° C. water bath for 10 min, and then in boiling water for 5 min to stop the reaction. After the addition of 1 ml of deionized water, the absorbance at 405 nm was measured by spectrophotometer (U-1800, Hitachi. Tokyo, Japan).
1. Determining the α-glucosidase inhibition activity of oleic acid (as the α-glucosidase inhibitor):
   A. Experimental group: enzyme+substrate+unsaturated fat acid (oleic acid)
   B. Control: enzyme+substrate+buffer
   C. Background: buffer+buffer+unsaturated fatty acid (oleic acid)
   Calculation of the rate of inhibition:

Inhibition rate %=[OD_{Control}−(OD_{Experimental group}−OD_{Background})]/OD_{Control}×100%

α-Glucosidase inhibition activity of oleic acid

| Concentration (mg/ml) | Inhibition rate (%) |
|---|---|
| 0.01 | 11.8 ± 0.99 |
| 0.02 | 52.2 ± 0.55 |
| 0.04 | 96.7 ± 0.06 |

The IC₅₀ value was obtained by interpolation from the above data: IC₅₀=0.022 mg/ml (defined as the oleic acid concentration to obtain 50% enzyme inhibitory activity).
2. Determining the α-glucosidase inhibition activity of linoleic acid (as the α-glucosidase inhibitor):
   A. Experimental group: enzyme+substrate+unsaturated fatty acid (linoleic acid)
   B. Control: enzyme+substrate+buffer
   C. Background: buffer+buffer+unsaturated fatty acid (linoleic acid)
   Calculation of the rate of inhibition:

Inhibition rate %=[OD_{Control}−(OD_{Experimental group}−OD_{Background})]/OD_{Control}×100%

α-Glucosidasc inhibition activity of lineleic acid

| Concentration (mg/ml) | Inhibition rate (%) |
|---|---|
| 0.0125 | 2.99 ± 0.36 |
| 0.0250 | 30.10 ± 0.54 |
| 0.0500 | 91.60 ± 0.62 |

The IC₅₀ value was obtained by interpolation from the above data: IC₅₀=0.033 mg/ml.
3. Determining the α-glucosidase inhibition activity of α-linolenic acid (as the α-glucosidase inhibitor):
   A. Experimental group: enzyme+substrate unsaturated fatty acid (α-linolenic acid)
   B. Control: enzyme+substrate+buffer
   C. Background: buffer+buffer+unsaturated fatty acid (α-linolenic acid)
   Calculation of the rate of inhibition:

Inhibition rate %=[OD_{control}−(OD_{Experimental group}−OD_{Background})]/OD_{Control}×100%

α-Glucosidase inhibition activity of α-linolenic acid

| Concentration (mg/ml) | Inhibition rate (%) |
|---|---|
| 0.04 | 31.6 ± 1.80 |
| 0.05 | 57.5 ± 2.39 |
| 0.06 | 81.0 ± 0.13 |

The IC₅₀ value was obtained by interpolation from the above data: IC₅₀=0.047 mg/ml.

Analysis of the enzyme inhibition activity of traditional α-glucosidase inhibitor (for example acarbose):
1. Determining the α-glucosidase inhibition activity of acarbose (as the α-glucosidase inhibitor):
   A. Experimental group: enzyme+substrate+acarbose
   B. Control: enzyme+substrate+buffer
   C. Background: buffer+buffer+acarbose
   Calculation of the rate of inhibition:

Inhibition rate % =[OD_{Control}−(OD_{Experimental group}−OD_{Background})]/OD_{Control}×100%

α-Glucosidase inhibition activity of acarbose

| Concentration (mg/ml) | Inhibition rale (%) |
|---|---|
| 0.5 | 13.9 ± 0.19 |
| 1.0 | 29.4 ± 0.30 |
| 2.0 | 52.2 ± 0.37 |

The IC₅₀ value was obtained by interpolation from the above data: IC₅₀=1.88 mg/ml.

### Example 2

In this Example, vegetable oils were used as the α-glucosidase inhibitor, and their enzyme inhibition activity was determined as following. The well-known vegetable oils include (for example) sunflower oil, soybean oil and the like.
1. Determining the α-glucosidase inhibition activity of sunflower oil (as the α-glucosidase inhibitor):
   A. Experimental group: enzyme+substrate+sunflower oil
   B. Control: enzyme+substrate+buffer
   C. Background: buffer+buffer+sunflower oil
   Calculation of the rate of inhibition:

Inhibition rate % =[OD_{Control}−(OD_{Experimental group}−OD_{Background})]/OD_{Control}×100%

α-Glucosidase inhibition activity of sunflower oil

| Concentration (mg/ml) | Inhibition rate (%) |
|---|---|
| 0.1 | 26.4 ± 0.69 |
| 0.2 | 46.3 ± 0.59 |
| 0.4 | 69.0 ± 0.13 |

The IC₅₀ value was obtained by interpolation from the above data C₅₀=0.253 mg/ml.
2. Determining the α-glucosidase inhibition activity of soybean oil (as the α-glucosidase inhibitor):
   A. Experimental group: enzyme+substrate+soybean oil
   B. Control: enzyme+substrate+buffer
   C. Background: buffer+buffer+soybean oil
   Calculation of the rate of inhibition:

Inhibition rate % =[OD_{Control}−(OD_{Experimental group}−OD_{Background})]/OD_{Control}×100%

α-Glucosidase inhibition activity of soybean oil

| Concentration (mg/ml) | Inhibition rate (%) |
|---|---|
| 0.125 | 17.0 ± 0.82 |
| 0.250 | 31.4 ± 1.09 |
| 0.500 | 71.9 ± 1.36 |

The IC₅₀ value was obtained by interpolation from the above data: IC₅₀=0.358 mg/ml.

The α-glucosidase inhibition activity of vegetable oils can be improved by hydrolysis of vegetable oils to release the unsaturated fatty acids.

In the following hydrolytic method, vegetable oils (sunflower oil and soybean oil) were reacted with lipase (Candida rugosa, EC 3.1.1.3), which. was purchased from Sigma-Aldrich Co. (Saint Louis, Mo., USA). The hydrolytic method used in this example was described previously by Khor et al (Khor H T, Tan N H, Chua C L. Lipase-catalyzed hydrolysis of palm oil. Journal of the American Oil Chemists' Society 1986. 63:538-540); and Okada & Morrissey (Okada T. Morrissey M T. Production of n-3 polyunsaturated fatty acid concentrate from sardine oil by immobilized Candida rugosa lipase. Journal of Food Science 2008, 73:C146-150). Briefly, 0.05 ml of sample was added to 2.5 ml of the lipase (2800 U./ml; in 100 mM phosphate buffer, pH 6.9). The mixture was incubated in 37° C. water bath for 90 min, and then 2.5 ml of dichloromethane was added to extract the hydrolytic product (free fatty acids). The organic phase was isolated for further analysis.

IC₅₀ value of α-glucosidase inhibition by
the vegetable oils before and after hydrolysis

IC₅₀ (mg/ml)

| Vegetable oil | Before hydrolysis | After hydrolysis |
|---|---|---|
| Soybean oil | 0.358 | 0.035 |
| Sunflower oil | 0.253 | 0.028 |

The hydrolytic product of sunflower oil contained abundant free fatty acids, in which the content of oleic acid (with highest inhibition activity) was up to 763 mg/g, and the content of linoleic acid was 109 mg/g. The hydrolytic product of soybean oil contained 329 mg/g of linoleic acid (higher than that in sunflower oil), while the content of oleic acid was only 303 mg/g, thus the inhibition activity of soybean oil after hydrolysis was slightly lower than sunflower oil hydrolytic product. These results showed that the content of oleic acid and linoleic acid has positive correlation to the inhibition activity.

### Example 3

In this example, the α-glucosidase inhibition of hexane extracts of medicinal fungi were determined and compared with the α-glucosidase inhibitor of the invention. The well-known medicinal fungi include (for example) Maitake mushrooms (Grifolo frondosa), Hericium erinaceum, Agaricus blazei, Ganoderma lucidum, Coriolus versicolor, Phellimus lintens, Inonotus obliquus and the like. The difference of fatty acids contained in the hexane extract of vegetable oil and medicinal fungi is that: fatty acids in vegetable oil exist in the form of triglycerides; whereas fatty acids in medicinal mushrooms exist in a form of free fatty acid. The analytical process was the same as described as in vegetable oils.

IC₅₀ value of α-glucosidase inhibition
by the medicinal mushrooms (mg/ml)

| Sample | IC₅₀ (mg/ml) |
|---|---|
| Grifola frondosa | 0.038 |
| Hericium erinaceum | 0.039 |
| Agaricus blazei | 0.053 |
| Ganoderma lucidum | 0.077 |
| Coriolus versicolor | 0.125 |
| Phellimus linteus | 0.165 |
| Inonotus obliquus | 0.302 |
| Oleic acid | 0.022 |
| Linoleic acid | 0.033 |
| α-Linolenic acid | 0.047 |

The content of major free fatty acids in medicinal mushrooms and vegetable oils

| | Content of major free fatty acids (mg/g , dw) | | | | | |
|---|---|---|---|---|---|---|
| | C16:0 | C16:1 | C18:0 | C18:1 | C18:2 | C18:3 |
| Sample | (palmitic acid) | (palmitoleic acid) | (stearic acid) | (oleic acid) | (linoleic acid) | (α-linolenic acid) |
| Grifola frondosa | 65.0 ± 0.65 | tr* | 8.59 ± 0.25 | 445 ± 14.8 | 144 ± 2.09 | nd |
| Hericium erinaceum | 137 ± 1.30 | tr | 66.9 ± 1.89 | 151 ± 3.49 | 111 ± 2.54 | nd |
| Agaricus blazei | 39.1 ± 0.81 | tr | 15.7 ± 0.88 | 4.54 ± 0.39 | 229 ± 1.92 | nd |
| Ganoderma lucidum | 29.0 ± 0.76 | tr | 5.39 ± 0.14 | 166 ± 4.11 | 43.7 ± 0.38 | nd |
| Coriolus versicolor | 31.8 ± 0.84 | tr | 3.56 ± 0.08 | 28.2 ± 0.33 | 61.9 ± 2.27 | 5.30 ± 0.19 |
| Phellinus linteus | 18.6 ± 0.40 | tr | 4.17 ± 0.18 | 13.6 ± 0.19 | 25.0 ± 0.54 | nd |
| Inonotus obliquus | tr | tr | 0.51 ± 0.16 | 1.27 ± 0.23 | 1.66 ± 0.18 | nd |
| Hydrolyzed soybean oil | 36.7 ± 0.91 | nd** | 2.76 ± 0.08 | 303 ± 16.0 | 329 ± 12.2 | 55.2 ± 0.79 |
| Hydrolyzed sunflower oil | nd | nd | nd | 763 ± 35.5 | 109 ± 2.52 | nd |

*tr = trace
**nd = not detected

By the IC₅₀ value (defined as the inhibitor concentration to reach 50% enzyme inhibitory activity) obtained from an unsaturated fatty acid of the invention, compared with vegetable oils, acarbose and medicinal fungi as listed above, the unsaturated fatty acid of the invention, such as oleic acid, linoleic acid and α-linolenic acid obtained 50% enzyme inhibitory activity at the concentration of 0.022 mg/ml, 0.033 mg/ml and 0.047 mg/ml, respectively.

Therefore, the unsaturated fatty acid of the invention used as an α-glucosidase inhibitor may inhibit the absorption of carbohydrate and prevent the occurrence of postprandial hyperglycemia. In another word the unsaturated fatty acid of the invention acts on inhibiting α-glucosidase activity to delay the conversion of disaccharides to monosaccharides, and further to achieve the effect of lowering blood glucose. Accordingly, the α-glucosidase inhibitor of the invention will be a potential hypoglycemic agent for preventing or treating Type 2 diabetes mellitus, and for use as an antiobesity drug.

From the above description, the unsaturated fatty acid of the invention is used as an α-glucosidase inhibitor as well as a essential nutrient for human body. The unsaturated fatty acid of the invention will not produce side effects to human body, and have a better efficacy than traditional oral hypoglycemic agents.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions, Thus, other embodiments are also within the claims.

What is claimed is:

1. A α-glucosidase inhibitor, which is composing of an unsaturated fatty acid composition.

2. The α-glucosidase inhibitor of claim 1, wherein the unsaturated fatty acid composition comprising the extract from mushroom.

3. The α-glucosidase inhibitor of claim 1, wherein the unsaturated fatty acid composition comprising the extract from edible vegetable or the hydrolytic product of solvent-extracted vegetable oil obtained by lipase.

4. The α-glucosidase inhibitor of claim 1, wherein the unsaturated fatty acid composition is a monounsaturated fatty acid composition.

5. The α-glucosidase inhibitor of claim 4, wherein the unsaturated fatty acid composition comprising oleic acid.

6. The α-glucosidase inhibitor of claim 1, wherein the unsaturated fatty acid composition is a polyunsaturated fatty acid composition.

7. The α-glucosidase inhibitor of claim 6, wherein the unsaturated fatty acid composition comprising linoleic acid.

8. The α-glucosidase inhibitor of claim 6, wherein the unsaturated fatty acid composition comprising α-linolenic acid.

9. The α-glucosidase inhibitor of claim 1, wherein the unsaturated fatty acid composition is a combination of monounsaturated fatty acid and polyunsaturated fatty acid composition.

10. The α-glucosidase inhibitor of claim 9, wherein the content of the monounsaturated fatty acid is higher than the polyunsaturated fatty acid.

11. The α-glucosidase inhibitor of claim 9, wherein the content of the monounsaturated fatty acid is lower than the polyunsaturated fatty acid.

12. The α-glucosidase inhibitor of claim 9, wherein the content of the monounsaturated fatty acid is equal to the polyunsaturated fatty acid.

13. The α-glucosidase inhibitor of claim 1, wherein the unsaturated fatty acid composition is used for preventing or treating diabetes.

14. The α-glucosidase inhibitor of claim 2, wherein the unsaturated fatty acid composition is used for preventing or treating diabetes.

15. The α-glucosidase inhibitor of claim 3, wherein the unsaturated fatty acid composition is used for preventing or treating diabetes.

16. The α-glucosidase inhibitor of claim 4, wherein the unsaturated fatty acid composition is used for preventing or treating diabetes.

17. The α-glucosidase inhibitor of claim 6, wherein the unsaturated fatty acid composition is used for preventing or treating diabetes.

18. The α-glucosidase inhibitor of claim 9, wherein the unsaturated fatty acid composition is used for preventing or treating diabetes.

19. The α-glucosidase inhibitor of claim 10, wherein the unsaturated fatty acid composition is used for preventing or treating diabetes.

20. The α-glucosidase inhibitor of claim 11, wherein the unsaturated fatty acid composition is used for preventing or treating diabetes.

21. The α-glucosidase inhibitor of claim 12, wherein the unsaturated fatty acid composition is used for preventing or treating diabetes.

22. The α-glucosidase inhibitor of claim 1, wherein the unsaturated fatty acid composition is used for preventing or treating obesity.

23. The α-glucosidase inhibitor of claim 2, wherein the unsaturated fatty acid composition is used for preventing or treating obesity.

24. The α-glucosidase inhibitor of claim 3, wherein the unsaturated fatty acid composition is used for preventing or treating obesity.

25. The α-glucosidase inhibitor of claim 4, wherein the unsaturated fatty acid composition is used for preventing or treating obesity.

26. The α-glucosidase inhibitor of claim 6, wherein the unsaturated fatty acid composition is used for preventing or treating obesity.

27. The α-glucosidase inhibitor of claim 9, wherein the unsaturated fatty acid composition is used for preventing or treating Obesity.

28. The α-glucosidase inhibitor of any one of claim 10, wherein the unsaturated fatty acid composition is used for preventing or treating obesity.

29. The α-glucosidase inhibitor of claim 11, wherein the unsaturated fatty acid composition is used for preventing or treating obesity.

30. The α-glucosidase inhibitor of claim 12, wherein the unsaturated fatty acid composition is used for preventing or treating obesity.
